(19) United States
(12) Patent Application Publication
(10) Pub. No.: US 2005/0265945 A1
(43) Pub. Date: Dec. 1, 2005

(54) Method for thickening hair and hair thickener

(76) Inventor: Sanford E. Schnoll, San Francisco, CA (US)

Correspondence Address: SCHNECK & SCHNECK, P.O. BOX 2-E, SAN JOSE, CA, 95109-0005, US

(21) Appl. No.: 10/854,692

(22) Filed: May 25, 2004

(51) Int. Cl. A61K 7/06; A61K 7/11; A61K 9/14

(52) U.S. Cl. 424/70.14

(57) ABSTRACT

Particles of human hair, each particle having no dimension greater than 3 mm, are prepared and affixed to a desired region, which may or may not have hair, to achieve thickening of the hair on the region or to add hair to the region, if lacking in hair. Such regions include the scalp or eyebrow. The particles of human hair may be prepared by cutting hair or by snap freezing and pulverizing hair into the desired size. The hair particles may be combined with a carrier, such as a hair styling agent, for affixing.

### FIELD OF THE INVENTION

The present invention relates to natural human hair thickener and methods for preparing and affixing natural hair thickeners.

### BACKGROUND OF THE INVENTION

Hair loss is a common problem for many men and women. As a consequence, systems and methods to supplement regions of thinning hair have been developed.

In some systems and methods, synthetic fibers are utilized to supplement regions of thinning hair. U.S. Pat. No. 5,005,596 to Yamada, discloses artificial hair made of a synthetic filament with silver particles applied to the filament by vacuum deposition. The artificial hair is used for hair implantation.

In other systems and methods, fibers comprised of the same organic keratin protein as natural hair are sprayed onto a scalp to supplement regions of thinning hair. Animal fibers, such as ground-up sheep's wool, are utilized.

In additional systems and methods, natural human hair may be utilized to supplement thinning hair. U.S. Pat. No. 5,575,298 to Hinton discloses use of human hair particles on a layer to join a hair supplement to the scalp. The layer includes an adhesive on a lower surface of the layer which is used for attachment to human hair on the scalp.

Natural human hair is a complex structure comprised of a hair shaft including a cortex, a cuticle, microfibrils and macrofibrils. Microfibrils and macrofibrils are composed of keratin. Use of natural human hair is desirable, as it typically appears more natural looking than synthetic hairs, keratin protein, or animal hairs. Though keratin protein or animal hairs may have a protein in common with natural human hair, they are lacking in remaining structural elements of natural human hair.

It is an object of the present invention to provide a method and composition for increasing the thickness of hair.

It is another object of the present invention to increase thickness of hair.

It is an additional object of the present invention to provide a method and composition concealing hair loss.

### SUMMARY OF THE INVENTION

These and other objects have been achieved by preparing a composition of particles of natural human hair, each of the particles ranging in size from 3 mm or less, and affixing the human hair particles to human hair located on a desired region of the human body, such as a scalp or an eyebrow. Additionally, or alternatively, the particles adhere to a surface lacking in hair, including a scalp. Because the particles are comprised of natural human hair, the particles blend in naturally with the existing hair while thickening a person's hair. Further, the small size of the particles allows a person to gradually build on the thickness of adhered particles and/or the hair to be supplemented resulting in a natural looking, desired amount of hair. The user may utilize particles of approximately the same size or of varied size to build on the thickness of existing hair to a desired level and/or to build the thickness of the particles to a desired level. The human hair particles may be comprised of a desired type or color.

The human hair particles, in one embodiment, are prepared by cutting hair into the specified size. In another embodiment, the particles are prepared by snap freezing human hair particles in liquid nitrogen and by pulverizing the frozen particles into the specified size. Pulverizing includes one or more methods for reducing the size of the hair such as crushing or grinding, as well as other methods. The particles may be in powder or granular form.

Fixing agents may be applied to human hair particles to affix the hair particle to the desired location. In one example, the human hair particles are combined with a carrier, such as a hair styling agent for affixation.

Also, the human hair particles may be adhered to hair supplements, such as hair pieces, to thicken the appearance of the hair on the hair pieces. The hair pieces may be comprised of, for example, synthetic hair.

### BRIEF DESCRIPTION OF THE DRAWINGS

FIG. 1 is a perspective view of human hair particles of the present invention.

FIG. 2 is a perspective view of hair, on a portion of a surface, prior to attachment of the hair particles of FIG. 1.

FIG. 3 is a perspective view of human hair particles of FIG. 1 attached to the hair and surface of FIG. 2.

### DETAILED DESCRIPTION OF THE INVENTION

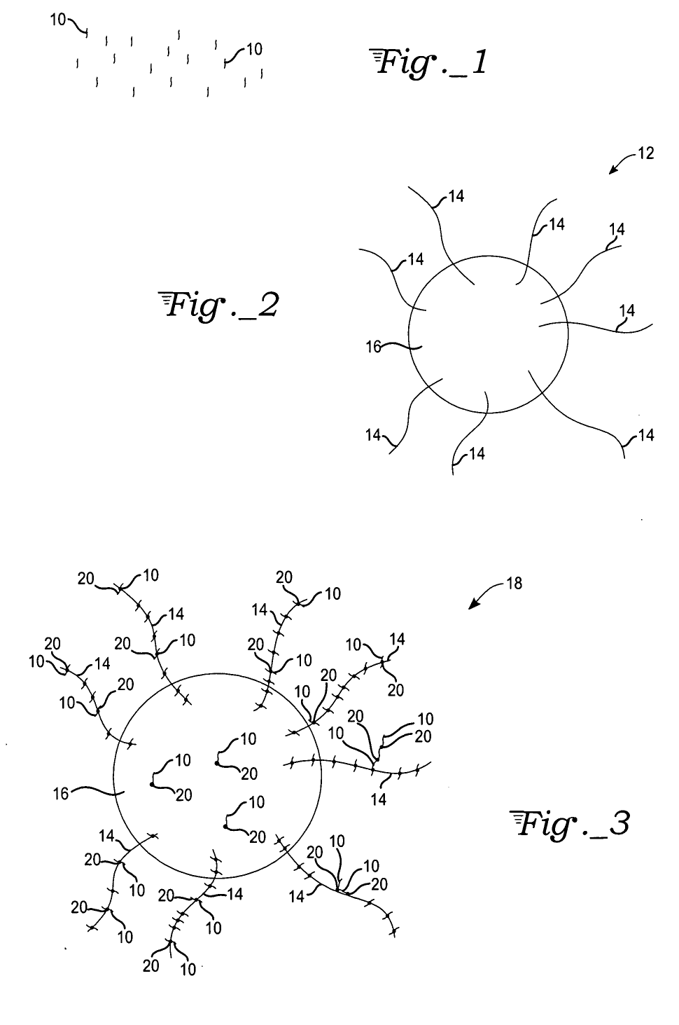

FIG. 1 illustrates human hair particles 10 of the present invention. The human hair particles 10 of the present invention range in size from 3 mm or less. The particles 10 may be small enough to comprise a powder or to be granular in form. No dimension of the human hair particles 10 is greater than 3 mm. For example, where the particles 10 are round, no diameter is greater than 3 mm and where the particles are straight, as depicted, no length or width of the particle 10 is greater than 3 mm. Though only one shape and type of particles 10 is illustrated, the particles 10 may vary in shape and type and may include a desired mixture of shapes and types. Hair types include for, example, curly or straight. Further, the human hair particles 10 are comprised of a desired color or mixture of colors.

In one example, a method for harvesting the human hair particles 10 comprises cutting human hair to a particle size as described above. Scissors or other equipment may be used to cut hair in to the desired particle size. The human hair to be cut may be obtained from the person whose hair is to be thickened or it may be obtained from other sources, such as a business that specializes in the sale of human hair.

In another method for harvesting the human hair particles 10, human hair is snap frozen in liquid nitrogen. The human hair to be frozen may be obtained from the person whose hair is to be thickened or it may be obtained from other sources. The snap frozen human hair is pulverized into human hair particles 10 of the present invention. In one example of preparing particles 10, hair is cut into pieces approximately 5 mm in length. The cut hair pieces are mixed with liquid nitrogen at, for example, −196 degrees Celsius. The frozen hair is then pulverized into particles 10 of the present invention. In one example, pulverizing the frozen hair includes crushing the hair with a mortar and pestle. In another example, pulverizing includes crushing the frozen hair with a mechanical mortar and pestle. In another example, pulverizing includes grinding.

Once the hair is pulverized into particles 10 of the present invention, the particles may be separated by size. A sizing sieve may be utilized to separate by size. Particles 10 may be divided into particles of the same size or of different sizes, depending on the user's preference.

A region 12, which is desired to be supplemented with hair particles 10, includes hair shafts 14 and a surface 16, as illustrated in FIG. 2. In one example, the surface 16 is a body surface and the hair 14 which is desired to be supplemented with hair particles 10 comprises human hair on the body surface 16. For example, the surface 16 may comprise a human scalp and the hairs 14 to be supplemented may comprise human hairs on the scalp. In another example, the surface 16 may comprise a brow region of a head and the hairs 14 to be supplemented in thickness may include hairs of an eyebrow on the brow region.

In another example, the hair 14 which is desired to be supplemented is a part of a hair piece. The hair piece may include artificial hair (synthetic or animal hair) and the surface 16 may be a synthetic surface.

A region 18 supplemented with hair particles 10 is illustrated in FIG. 3. The human hair particles 10 of the present invention may be affixed to the hair 14 to be supplemented or the desired surface 16 resulting in a thickening of the hair 14. Further, the human hair particles 10 may be affixed or to other particles 10. A fixing agent 20 is depicted in FIG. 3, affixing the human hair particles 10 to the hair 14 to be supplemented, to hair particles 10 which have been affixed to human hair particles 10, to hair particles 10 which have been affixed to the hair 14, and to the surface 16.

The small size of the particles 10 of the present invention allow a person to gradually build on the thickness of the hair 14 to be supplemented resulting in a natural looking, desired amount of hair. Further, the particles 10 may build on other particles 10 which have already been affixed to the hair 14 to be supplemented or to the surface 16 for a natural look. The user may utilize particles 10 of approximately the same size or of a varied size to build on the thickness of existing hair 14 or particles 10 to a desired level. Also, the particles 10 conceal regions lacking in hair.

The user may use hair particles 10 which are the same color as the hair 14 to be supplemented, or the user may use hair particles 10 which are different in color from the hair 14 to be supplemented in order to change the color of the hair 14 to be supplemented.

The hair particles 10 of the present invention are affixed to a desired surface, to human hair disposed on the body surface, to other particles 10, and to hair on synthetic surfaces in various ways. A fixing agent 20 is used to affix the hair particles 10 to the hair 14 to be supplemented, to hair particles 10, and/or to the surface 16. For example, the hair particles 10 of the present invention are dispersed within a carrier, such as a hair styling product and the carrier, including the particles 10, is applied to the hair 14 to be supplemented. Hair styling products include, for example, mousse, cream, gel, pomade, and spray. The carrier acts as the fixing agent 20 or adhesive. In one example, hairspray or other fixative may be applied in addition to the fixing agent 20.

In another example, the human hair particles 10 are applied to the hair 14 to be supplemented, the human hair particles 10 which have already been affixed to hair 14, or surface 16, and/or to the desired surface 16, and are affixed with the fixing agent 20, such as hairspray. The hairspray is sprayed onto the human hair particles 10, hair 14, and/or surface 16 to affix the human hair particles 10. Hairspray may be sprayed onto human hair particles 10 of the present invention, onto hair 14, or onto surface 16 after the particles have been applied to hair 14 which is desired to be thickened, to affixed particles 10, and/or to surface 16. Hairspray acts as the fixing agent 20 or adhesive which affixes the human hair particles 10 of the present invention to the surface 16, hair 14, and/or particles 10 on the surface 16.

In one example, the particles 10 may be applied to surface 16, hair 14, and/or particles 10 on the surface 16 without a fixing agent.

The drawings are for illustrative purposes only and are not intended to limit the invention. In particular, the drawings are not intended to limit the location, orientation, or amount of the fixing agent 20 or the human hair particles 10 of the invention.

1. A method for thickening human hair, comprising:
harvesting particles of human hair, each of said particles ranging in all dimensions from 3 mm or less; and
affixing said particles of human hair to human hair.

2. The method of claim 1 further comprising applying said particles of hair to said human hair and affixing said particles with hairspray.

3. The method of claim 1 further comprising combining said particles with a carrier before affixing said particles to human hair.

4. The method of claim 3 wherein said carrier is selected from a group of carriers consisting of mousse, cream, gel, pomade, and spray.

5. The method of claim 3 wherein said carrier is a hair styling product.

6. The method of claim 1 wherein said human hair is located on a human scalp.

7. The method of claim 1 wherein said human hair is located on a brow region.

8. The method of claim 1 wherein said human hair is disposed on a body surface.

9. The method of claim 1 further comprising harvesting said particles of human hair ranging in size from 3 mm or less by cutting human hair.

10. The method of claim 1 wherein said human hair particles are the same color as said hair to which said human hair particles are affixed.

11. The method of claim 1 wherein said human hair particles are a color different from a color of said hair to which said human hair particles are affixed.

12. The method of claim 1 further comprising harvesting said particles of human hair by snap freezing human hair in liquid nitrogen, and pulverizing said human hair into particles.

13. The method of claim 12 wherein pulverizing includes grinding.

14. The method of claim 12 wherein pulverizing includes crushing.

15. The method of claim 1 wherein said particles of human hair are a powder.

16. The method of claim 1 wherein said particles of human hair are granular.

17. The method of claim 1 further comprising harvesting particles of human hair of a desired color.

18. The method of claim 1 further comprising harvesting particles of human hair of a desired type.

19. The method of claim 1 further comprising harvesting a selected dimension of particle.

20. The method of claim 19 wherein said dimension is a length.

21. The method of claim 19 wherein said dimension is a diameter.

22. The method of claim 1 wherein said particles are round.

23. A method for adding hair, comprising:
harvesting particles of human hair having dimensions, said particle having no dimension greater than 3 mm; and
affixing said particles of human hair to a desired location.

24. The method of claim 23 wherein said desired location is a scalp.

25. The method of claim 23 wherein said desired location is a brow region.

26. The method of claim 23 wherein said desired location comprises a hair piece.

27. The method of claim 26 wherein said hair piece includes a surface to which artificial hairs are attached and to which said particles are affixed.

28. A hair product for thickening hair, comprising:
particles of human hair having dimensions, said particles having no dimension greater than 3 mm; and
a carrier within which said particles are dispersed.

29. The hair product of claim 28 wherein said carrier is a hair styling product.

30. The hair product of claim 28 wherein said carrier is selected from a group of carriers consisting of mousse, cream, gel, pomade, and spray.

31. The hair product of claim 28 wherein said particles are in a powder form.

32. The hair supplement of claim 28 wherein said carrier is a fixing agent.

33. The hair supplement of claim 28 wherein said particles are granular.

34. A method for thickening human hair, comprising:
harvesting particles of human hair, each of said particles ranging in all dimensions from 3 mm or less; and
applying said particles of human hair to a desired surface.

35. A method for supplementing a region with hair, comprising:
applying particles of human hair to a desired region on a human body, said particles of human hair having an individual particle size ranging in dimensions from 3 mm or less.

36. The method of claim 35 wherein said desired region includes human hair affixed to said body, said applying of said particles of human hair thereby thickening said human hair affixed to said body.

37. The method of claim 35 further comprising affixing said particles of human hair to said desired region on said body.

38. A composition comprising:
particles of hair having an individual particle size ranging in dimensions from 3 mm or less, said particles previously frozen; and
a carrier within which said particles of hair are dispersed.

39. A composition comprising:
particles of human hair having an individual particle size with dimensions of 3 mm or less; and
a carrier within which said particles are dispersed.
